(19) United States
(12) Patent Application Publication
(10) Pub. No.: US 2008/0274565 A1
(43) Pub. Date: Nov. 6, 2008

(54) Method for the quantitative measurement of analytes in a liquid sample by immunochromatography

(75) Inventors: Hamidou Samake, Paris (FR); Nathalie Martineau, Asnieres Sur Seine (FR); Muriel Gonidec, Chantilly (FR)

Correspondence Address: SUGHRUE MION, PLLC, 2100 PENNSYLVANIA AVENUE, N.W., SUITE 800, WASHINGTON, DC, 20037, US

(73) Assignee: DIAGNOSTICA STAGO, Asnieres Cedex (FR)

(21) Appl. No.: 11/819,235

(22) Filed: Jun. 26, 2007

Related U.S. Application Data
(63) Continuation of application No. PCT/FR2006/000236, filed on Feb. 2, 2006.

(30) Foreign Application Priority Data
Feb. 4, 2005 (FR) 0501145
Sep. 6, 2005 (FR) 0509097

(51) Int. Cl. G01N 33/543; B01J 19/00

(52) U.S. Cl. 436/518; 422/56

(57) ABSTRACT

The invention concerns a method for the quantitative measurement of at least one analyte of interest in a liquid sample by immunochromatography, said method comprising weighted measurement of the quantity of analyte with respect to a control.

The method is of particular application to medical diagnostics in the field of hemostasis, for example to exclude a risk of diagnosis of venous thromboembolic disease.

The present invention relates to a method for the quantitative measurement of at least one analyte of interest in a liquid sample by immunochromatography, and to the device developed for this purpose.

Appearing towards the end of the 1980s, immunochromatography is a technique which has seen tremendous advances. It currently has a wide range of applications from the traditional fields of medical or veterinary diagnostics to the environment or to agroalimentary fields.

Immunochromatography falls into the group of diagnostic methods based on affinity binding reactions between specific bound pair members, collectively known under the generic term of immunoassay.

Generally, immunoassays are divided into two major categories depending on the approach employed: those involving competition for a specific ligand between the investigated analyte and a tagged analyte and those termed “sandwich” techniques in which binding of the analyte to a first specific ligand is revealed by a tagged second specific ligand fixed on said analyte.

Immunoassays have steadily evolved into devices which are becoming ever simpler to use, allowing the development of routine diagnostic methods which are rapid and moderately priced.

That development has been particularly important in the medical field with the emergence of “point of care” or “home testing” diagnostics in which diagnosis is carried out at the patient's bedside or residence without the need for automated laboratory analyses.

Immunochromatography falls into the group of techniques used for that type of rapid diagnostics.

Immunochromatography is a solid phase diagnostic method using dry chemistry and lateral migration over an inert membrane. This approach employs a porous support in the form of a strip into which are integrated, in the dry form, the reagents necessary for conducting the test. The membrane is treated so that a recognition reaction between specific bound partners (generally antigen/antibody) occurs at a predetermined zone and can be revealed at that location. Schematically, when the sample to be analyzed is deposited on the support, it migrates along the membrane by capillary action. The investigated analyte is captured by a specific ligand immobilized on a predetermined zone of the membrane, and the reaction is revealed by a tagged second specific ligand using the sandwich method.

It is also possible to use a competition type reaction in immunochromatography. In that case, a tagged analyte that competes with the investigated analyte is used to bind with the immobilized ligand. For that type of test, it is also possible to use the tagged ligand: in that case, the investigated analyte is in competition with the immobilized analyte.

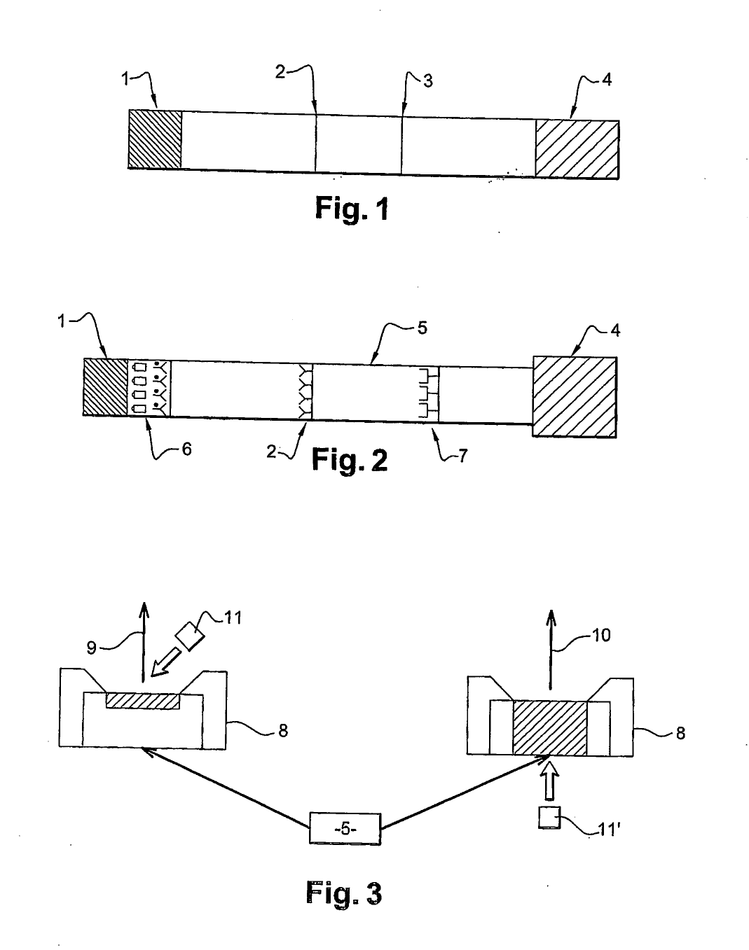

Finally, it is also possible to use a serological type reaction when investigating an antibody by immunochromatography. In this case, assay is possible. The antigen specific to the disease is immobilized on the support. It can capture human antibodies present in the sample that are specific to the antigen and the disease. The presence of human antibodies is revealed after adding a secondary antibody directed against human immunoglobulins. For such serological tests, it is even possible to type the antibodies to determine the class of human antibodies detected (IgM, IgG or IgA) (The principle of an immunochromatography strip is shown in FIG. 1).

Several prior art documents describe immunochromatographic tests and devices and particular adaptations of this technique to certain applications. A non-exhaustive list of documents which can be cited are United States documents U.S. Pat. No. 5,591,645, U.S. Pat. No. 5,120,643 and European patents EP-A-0 299 428 or EP-A-0 250 137 which describe the general principle of immunochromatographic tests comprising a test strip allowing migration of liquid by capillary action and capture of the investigated analyte by a specific reagent immobilized in a predetermined zone on the strip, EP-A-0 291 184 which describes a specific analytical apparatus composed of a hollow case containing a reaction support, EP-A-0 383 619 which concerns a specific membrane support, and EP-A-0 267 006 which describes a qualitative immunochromatographic method for simultaneous detection of the presence of several analytes in a sample.

However, the majority of rapid unitary tests involving immunochromatography described in the prior art are qualitative yes/no type tests based on reading with the naked eye. However, the subjectivity of reading in that manner is a source of variability and confusion. Further, such tests cannot be used in certain applications for which a decision cut-off exists as is the case, for example, with certain exclusion markers for thrombosis, such as D-dimers, in the hemostasis field.

Thus, the need for approaches that can allow the results obtained to be quantified as simply as possible has rapidly arisen.

One of the oldest current approaches is that using a specific internal reference termed the procedural control zone (PCZ) (see, for example, EP-A-0 542 627, International patent application WO-A-96/0179, WO-A-94/07163 or AN, C D, YOSHIKI T, LEE G and OKADA Y, Cancer Letters 2001, 162: 135-139, CHAN, C P Y, SUM K W, CHEUNG K Y, GLATZ J F C, SANDERSON J E, HEMPEL A, LEHMANN M, RENNEBERG I and RENNEBERG R, J Immunol Methods, 2003, 279: 91-100).

Schematically, in that system, the sample to be analyzed is brought into the presence of a specific conjugate for the investigated analyte during deposition on the immunochromatography strip.

The conjugate binds to the investigated analyte and the complex formed migrates along the membrane to the specific test capture zone for the analyte. The coloration which develops at this zone is proportional to the concentration of investigated analyte. The excess conjugate which has not been bound to the analyte is captured at a PCZ zone downstream of the test capture zone on the strip. That PCZ zone comprises an immobilized reagent specific to the conjugate employed. In practice, with that approach, the conjugate is in general a monoclonal antibody of animal origin and the reagent immobilized in that PCZ zone is an anti-species antibody directed against the animal species of the conjugate.

Although it is relatively simple in principle, that system suffers from certain disadvantages:—the same conjugate reagent acts to reveal the test and the control. For this reason, the PCZ signal is greatly dependent on the concentration of analyte. For a high concentration, the signal can be greatly reduced or even absent in extreme cases.

Further, in such a control system, there is no weighting of variations linked to the sample and to the membrane.

In other approaches, EP-A-0 088 636 describes a quantitative analytical method based on the use of a plurality of successive reaction zones specific to the same analyte spaced out on the migration support.

WO-A-91/12528 describes a quantitative immunochromatographic method also using a multiple zone strip in which, schematically, the investigated analyte is sandwiched between two specific reagents one of which is coupled to a tag and the other to biotin. The product resulting from said interactions migrates along the strip and is captured at the detection zone by a capture agent constituted by avidin fixed to latex particles, all of them being immobilized on the strip.

WO-A-97/09620 concerns a method for quantitative or semi-quantitative detection of an analyte in a sample, consisting of using a calibration agent and based on the presence of a plurality of zones at least one of which is for calibration, one for measurement and a final zone to check that the test is functioning properly.

WO-A-00/43786 describes an approach for quantifying results obtained by immunochromatography, displaying results associated with coloration, in which values or ranges of values are applied to signal colorations or coloration intensities, and in which the coloration of the result is compared with that of an internal calibration reference. The method employed in that reference is in reality that of a semi-quantitative test, defining a reference coloration intensity corresponding to a reference concentration of the investigated analyte. It does not use a measuring instrument and pre-calibration, the only means for guaranteeing reliable results with quantitative tests.

WO-A-97/37222 and WO-A-02/077646 concern quantitative immunochromatographic methods carried out using a RAMP™ (rapid antigen measurement platform) device. Those methods are relatively complex application-wise. They involve differentiating between the application zone for the sample and the zone bringing it into contact with the reagent specific to the investigated analyte. Further, they use populations of particles capable of binding specifically to the desire analyte as the detecting reagent, which populations migrate with the latter to a detection zone.

The present invention aims to provide a simple quantitative general immunochromatographic method ensuring reliable results despite certain fluctuations due to factors such as the sample (viscosity, composition etc) or the membrane (micro-heterogeneity of the structure), using a specific weighting system.

In particular, the invention proposes means allowing to collect a signal related to the presence of an analyte which is sought and also a control signal, the latter being chosen to be independent of the main reaction involving the analyte and thus to ensure a constancy of the control signal and thus the objectivity of the test result.

Thus, the present invention provides a method and device of this type in which a biological sample is deposited on one end of a strip of porous material and migrates by capillary action towards the second end of the strip, the sample coming into contact at the beginning of its migration with a test product (capture reagent) which can form a specific binding complex with the substance (analyte) to be assayed and also with a control product (control reagent). The complex formed by the substance with the test product is displaced during migration of the sample towards the second end of the strip which comprises a test zone in which a capture reagent for that complex is deposited. The control product is also displaced by migration of the sample towards the second end of the strip which comprises a control zone in which a capture reagent for the control product has been deposited, the test zone being close to the control zone.

The test reagent and the control product comprise labels for detecting or marking of any type, which are detectable by optical, magnetic, electro magnetic or other means.

In a particular aspect, the present invention provides a method for the quantitative measurement of at least one analyte of interest in a liquid sample by immunochromatography, said method comprising the following steps:
a) providing an immunochromatography device comprising a solid support in the form of a strip, said support comprising a sample application zone, a migration zone, a specific capture zone for each of the investigated analytes and a ratiometric measurement control zone (RMC), the specific capture zone or zones each comprising a capture reagent immobilized on the support, said capture reagent forming a specific bound pair with one of the investigated analytes, and said RMC zone comprising a control capture reagent immobilized on the support, said reagent forming a specific bound pair with a control reagent carrying a detectable tag;
b) bringing the sample into contact with a control reagent carrying a detectable tag and with a conjugate or a mixture of conjugates, each conjugate being constituted by a reagent forming a specific bound partner with one of the investigated analytes and carrying a detectable tag absorbing at the same wavelength as the tag for the control reagent;
c) depositing the sample which has been brought into the presence of the control reagent and the conjugate or mixture of conjugates on the application zone of the support;
d) maintaining the device under conditions sufficient to allow the investigated analyte or analytes to form bound pairs with the specific conjugate or conjugates and to allow transport of the investigated analyte or analytes and the control reagent by capillary action along the solid support to each of the specific capture zones for the investigated analyte or analytes and to the RMC zone;
e) maintaining the device under conditions sufficient to allow further binding of the investigated analyte or analytes with the capture reagent or reagents at the capture zone or zones, and binding of the control reagent with the control capture reagent at the RMC zone;
f) measuring the intensity of the test signal produced by the conjugate bound to the investigated analyte at each of the capture zones and that of the RMC control signal produced by the control reagent at the RMC zone;

whereby the quantity (or ratio) of each analyte of interest in the sample is directly proportional to the ratio of each test signal to the RMC control signal.

The present invention also pertains to an immunochromatography device for the quantitative analysis of one or more analytes in a sample using the method of the invention, comprising a solid support in the form of a strip, said support comprising a sample application zone, a migration zone, a specific capture zone for each of the investigated analytes and a ratiometric measurement control zone (RMC), in which the RMC zone comprises a control capture reagent immobilized on the support, said reagent being capable of forming a pair by specifically binding with a control reagent carrying a detectable tag.

According to the present invention, the control reagent which migrates along the immunochromatography membrane and which is then captured by the control capture reagent immobilized in the RMC zone has no cross reactivity with the investigated analytes. It thus provides a signal the intensity of which is independent of the concentration of the investigated analyte in the sample. Also, the control capture reagent can be chosen not to have any interference, in particular any crossed reactivity in case of antibodies, with the other reagents of the dosage, in particular the one used for the capture of analytes.

The signal which develops in this zone thus validates the observed results by weighting the influences of certain experimental conditions, such as viscosity and the composition of the sample, variations in the physico-chemical properties of the membrane, etc.

The signals obtained using the method of the invention are advantageously measured using a signal reading apparatus which can provide numerical results and eliminate any subjectivity.

Within the context of the method of the invention, the conjugate or mixture of conjugates specific to the investigated analyte or analytes and the RMC control reagent may be added to the sample prior to deposition on the application zone of the support. In this implementation, the sample/conjugate/RMC reagent mixture is then deposited on the application zone in accordance with steps b) and c) of the method described above.

In a second preferred aspect of the invention, the conjugate or mixture of conjugates and the RMC control reagent are used in the dry form. They are then contained in a reservoir in the region of the sample application zone and contact of the liquid sample with the conjugate or mixture of conjugates and with the RMC control reagent occurs when the sample is deposited on the application zone of the support. Deposition of the sample will cause the conjugates and RMC control reagent to dissolve, cause the conjugate or conjugates to bind to their specific analyte(s) and cause migration along the support by capillary action of the complexes formed between each analyte and its specific conjugate, the RMC reagent and, possibly, excess conjugates which have not reacted with the analytes.

In one particular implementation of the invention, the sample is deposited on a reservoir which has been saturated (for example in a PBS-1% Regilait—5% sucrose saturation solution (in an amount of 0.5 ml to 1 ml/test) in the saturation bath).

In this particular variation, the invention provides: a method for the quantitative measurement of at least one analyte of interest in a liquid sample by immunochromatography, said method comprising the following steps:
a) providing an immunochromatography device comprising a solid support in the form of a strip, said support comprising a sample application zone, a migration zone, a specific capture zone for each of the investigated analytes and a ratiometric measurement control zone (RMC), said application zone comprising a reservoir containing a control reagent carrying a detectable tag and a conjugate or a mixture of conjugates, each conjugate being constituted by a reagent that is capable of forming a specific bound partner with one of the investigated analytes and carrying a detectable tag absorbing at the same wavelength as the control reagent tag, and said RMC zone comprising a control capture reagent immobilized on the support, said reagent forming a specific bound pair with the control reagent carrying a detectable tag;
b) depositing the sample on the application zone of the support;
c) maintaining the device under conditions sufficient to allow the investigated analyte or analytes to form bound pairs with the specific conjugate or conjugates and to allow transport of the investigated analyte or analytes and the control reagent by capillary action along the solid support to each of the specific capture zones for the investigated analyte or analytes and to the RMC zone;
d) maintaining the device under conditions sufficient to allow further binding of the investigated analyte or analytes with the capture reagent or reagents at the capture zone or zones, and binding of the control reagent with the control capture reagent at the RMC zone;
e) measuring the intensity of the test signal produced by the conjugate fixed to the investigated analyte at each of the capture zones and that of the RMC control signal produced by the control reagent at the RMC zone;
   whereby the quantity (or ratio) of each analyte of interest in the sample is directly proportional to the ratio of each test signal to the RMC control signal.

According to a first embodiment of the invention, an assay is carried out as follows: an operator deposits the sample to be analyzed on the first end of the strip, optionally with a migration buffer, then starts a clock. The strip is then placed in a device which can read the results and which is triggered by the operator when a predetermined time has elapsed from the time the clock is started. The results are read by detecting signals generated by the labels in the test and control zones of the strip, after which the ratio of the recorded signals is produced and the quantity of substance present in the liquid sample is determined from said ratio using a calibration curve or table. The ratio of the signals detected in the test and control zones cancels out variations due to certain characteristics of the sample (its nature, viscosity, protein or ionic content, etc) and of the strip (in particular micro-variations in its structure and components). The ratio of the signals detected in the test and control zones is essentially a function of the concentration of the substance to be assayed in the sample and is, to at least a first approximation, independent of said characteristics of the sample and strip.

However, it has been determined that the accuracy of the assays so produced depends a great deal on the operator who must always follow the same procedure for each assay in order to guarantee the quality and reliability of the result, which is not always possible in practice. In particular, if the operator starts the clock more or less quickly after depositing the sample and optional migration buffer on the strip, the assay results may vary relatively greatly.

The operator may also make a mistake, such as omitting to start the clock or not adding the migration buffer, which means that the assay has to be repeated when the error is discovered, generally by the absence of a result—a lot of time is thereby wasted.

For these reasons, the invention describes, according to another embodiment, a method and device for quantitative assay which improve the accuracy and reliability of the assay results.

According to this embodiment, the invention relates to a method and device for quantitative assay of the type cited above, which can prevent operator errors and ensure good repeatability and assay reliability.

To this end, it provides a method for quantitative assay by immunochromatography of a substance present in a liquid sample, conforming to the description of the present application and characterized in that it also consists of automatically detecting the passage of labels into a given zone of the strip at the start of sample migration, automatically starting counting of a predetermined period of time to detection of said passage of the labels and, at the end of said period of time, automatically commencing acquisition of signals generated by the labels in the test and control zones of the strip.

In particular, the concerned process for quantitative assay consists in bringing the liquid sample into contact with a conjugate which can react with the substance to be assayed to form a complex, and with a control product, the conjugate and the control product comprising detection labels, migrating the sample under capillary action with the conjugate and the control product in a strip of porous material comprising a test zone which contains a reagent which can react with the complex and a control zone which contains a reagent which can react with the control product, detecting the signals generated by the labels in the test zone and in the control zone, producing the ratio of said signals and deducing the quantity of substance present in the sample from said ratio, said process being furthermore characterized in that it also consists of automatically detecting the passage of labels into a given zone of the strip at the start of sample migration, automatically starting counting of a predetermined period of time to detection of said passage of the labels and, at the end of said period of time, automatically commencing acquisition of signals generated by the labels in the test and control zones of the strip.

The described process comprising the step of automatical detection can be performed having recourse to the different variants of the characteristics of the process for quantitative measure of analyte or of the device for the execution of this process.

According to this embodiment including an automatical signal detection, the method of the invention thus envisages starting the countdown when the sample has already migrated over a certain distance in the strip, i.e. after a preliminary phase during which perturbations connected with the assay reaction may occur, on deposition of the sample or the migration buffer on the strip or dissolution of the conjugate, or complex formation. Starting the countdown after this preliminary phase guarantees a constant migration time in the strip for all samples prior to acquisition of the results, which would not be the case if the countdown were to commence at the start or at the end of deposition of the sample or migration buffer or during dissolution of the conjugate or during complex formation.

Thus, the quality of the results and the reliability of the assay are considerably improved.

Results acquisition consists, in a preferred embodiment, in measuring the areas of the signals generated by the labels in the test and control zones, producing the ratio of these areas, and deducing from this ratio and a calibration curve or table the quantity of the substance present in the sample.

In accordance with a further characteristic of the invention, the labels have a light absorption peak at a given wavelength, and the method consists of illuminating the strip at this wavelength, measuring the intensity of the light reflected or diffused by the labels at this wavelength using at least one photodetector, and processing the signals emitted by said photodetector to obtain the areas of the signals generated by the labels.

Advantageously, said photodetector is associated with means for scanning the strip to capture signals generated by the labels and to determine the areas of said signals as a function of a scan distance.

The invention also pertains to an immunochromatography device for carrying out the method defined above, said device comprising a solid support in the form of a strip, said support comprising a sample application zone, a migration zone, a specific capture zone for each of the investigated analytes and a ratiometric measurement control zone (RMC), in which the application zone comprises a reservoir containing a control reagent carrying a detectable tag and a conjugate or a mixture of conjugates in the dry form, each conjugate being constituted by a reagent forming a specific bound partner with one of the investigated analytes and carrying a detectable tag absorbing at the same wavelength as the control reagent tag and the RMC zone comprises a control capture reagent immobilized on the support, said reagent forming a specific bound pair with the control reagent carrying a detectable tag. Said device is shown schematically in FIG. 2.

According to an implementation of the device of the invention, the membrane of the strip is made of a porous inert material in order to enable a capillary flux comprised between 50 and 150 sec/4 cm, in particular lower than 100 sec/4 cm, for example from 90 sec/4 cm. The size of the pores of the support forming the strip can be of 10 μm.

In a particular implementation of the invention, the conjugate capable of specifically reacting to form a bond with an analyte is an antibody, for example a monoclonal antibody directed against a first antigenic site of the analyte. The analyte capture reagent bound to the conjugate may also be an antibody, for example a monoclonal antibody directed against a second site of the analyte.

In a particular implementation of the invention, the RMC control capture reagent (or compound) is also an antibody, for example a polyclonal antibody.

The reagents forming the conjugate binding with the analyte and the control capture reagent can be deposited by vaporizating the strip.

Within the context of the method of the invention described above, the use of a PCZ becomes optional. The function of that type of control in this case is limited to verifying whether the specific conjugate for the investigated analyte has been complexed or whether an excess of unused conjugate remains following the reaction.

Providing conjugates in a reservoir can ensure on the one hand, a storage, on the other hand, a uniform transfer of conjugates and sample to the migration membrane. After adding the sample to the test device comprising the conjugate reservoir, the tracer dissolves. The tracer is “released” or “leached” from the reservoir onto the migration membrane. The remainder of the reactions resulting in detection and/or assay occurs normally under optimum conditions.

The materials used to act as a conjugate reservoir are nonwoven materials. The following can be cited: glass fibers (GF), which are the most routinely used, then cellulose filters and hydrophilic polyesters.

The conjugate reservoirs are generally pretreated, the treatment being prior saturation with saturating agents, namely proteins and/or surfactants. After said saturation followed by rapid drying at a temperature of 37° C. (in general), the conjugate is deposited by immersion or by printing.

The use of a reservoir allows the control of the quantity of the released conjugates, the speed of said release and of the migration of the conjugates on the membrane.

The mode of positioning the RMC zone on the support is not relevant to carrying out the method of the invention, provided that it is distinct from that of each of the capture zones. The separation between the RMC zones and the capture zones may be between 3 and 10 mm, advantageously about 5±0.5 mm. For simplification, the RMC zone is preferably located downstream of the other test capture zones.

The method of the invention may advantageously be carried out for simultaneous quantification of a plurality of analytes, each analyte then being stopped from migrating on the support at the specific capture zone, and revealed by a specific conjugate.

Quantification of the concentrations of each analyte is advantageously carried out with a single RMC. In this case it is sufficient to calculate the ratio of each of the test signals to the RMC signal.

As indicated above, the control reagent captured in the region of the RMC zone and the investigated analytes have no cross reactivity, which guarantees a RMC signal intensity which is independent of the concentration and nature of the investigated analytes.

As an example, in the case of an assay carried out on a sample of human origin, the RMC is selected from molecules of animal origin with no cross reactivity with the investigated analyte type.

Thus, the control reagent may be selected from certain immunoglobulins of animal origin, such as chicken immunoglobulins.

The capture reagent in this case is an antibody directed against the animal species of said control reagent, namely a chicken anti-immunoglobulin antibody.

The bound pair formed by the RMC control reagent and its specific capture reagent is preferably of the antigen/antibody type.

However, it may be constituted by other affinity partners, for example of the latex or liposome type, or pairs such as: avidin (streptavidin)/biotin, protein A/immunoglobulin (Ig), protein G/Ig, lectins/concanavalin A (Con A), anti-haptoglobin/hemoglobin.

The tag used on the conjugates and on the RMC reagent is generally of two types:
direct, such as particular tags (particles of gold, latex, carbon, polycarbonate) and colorants, colored dextran, fluorescent tracers, etc. These tags are detectable per se with no supplementary reaction;
indirect, such as enzymatic tags which require a revealing reaction the product of which is detected and/or measured.

Preferably, the tag on the conjugate or mixture of conjugates is identical to that used on the RMC control reagent. Nevertheless, it is possible to use different tags, provided that they absorb at the same wavelength, so that a correlation can always be established during measurement.

Preferably, the tag used is particulate in type, in this case particles of gold such as colloidal gold.

As already stated above, quantitative measurement of the concentration of the investigated analyte is advantageously carried out using a strip reader which can measure the test and RMC signals in arbitrary units, for example by reflectance or transmittance.

The final measurement which is referred to is the test signal/RMC ratio. Thus, a measured ratio corresponds to each test carried out.

Firstly, a standard curve is established by assay of a reference analyte at a plurality of concentrations. For a given analyte, this standard curve serves as a reference for determining the quantity of an analyte in the sample, corresponding to the measured test signal/RMC signal ratio. The values on the standard curve further correspond to the ratio of the signal obtained with the reference analyte at a given concentration to the unique RMC signal. The RMC signal is selected by the skilled person to establish the standard curve and corresponds to a value for the complex formed between the RMC capture reagent-RMC which produces a signal (the RMC signal) located in the rising portion of the signal/complex curve as opposed to a value which would be in the flat part of the curve.

Secondly, the ratio measured for an assay carried out is transferred to this standard curve. It is then possible to translate the measured ratio into the concentration of the investigated analyte.

All assaying of a sample is thus carried out by referring to a standard curve which is traced or memorized by the reader in the form of a suitable presentation (graph, barcode, etc).

The described device is advantageously realized in such a way that the solid support constituted by the strip is contained in a casing. The casing impacts on the flow of the sample at the level of the reservoir and of the membrane, by preventing leakages of liquids. It can moreover be adapted to the reading system.

The detection agent generally used in the immunochromatographic tests is colloidal gold, which has an absorption peak at about 535 nm. In this case, for detection, the reader may be equipped with two LED (light emitting diode) sources emitting at 530 nm.

For this use, the two types of light (transmitted and reflected) have different characteristics:
reflectance is more or less independent of the environment and the equipment used has no influence on the strip itself;
transmittance is highly dependent on the equipment and more particularly on the material into which the reactive strip is inserted. This means that measuring transmittance may be easier or harder depending on the nature and thickness of the material used.

The measurements of the reflected and transmitted light are proportional to the concentration of analyte captured by a specific reagent coupled to particles of colloidal gold (conjugate).

In the illustrations of the invention, reflectance is preferably used.

The invention also relates to a process for manufacturing the described device for measurement and a device obtained by this process comprising the following steps:
coating (or immobilizing) on a membrane enabling the flow of a liquid by capillarity, by means on the one hand, of the dosage reagent of the analyte assayed in a sample, on the other hand, a capture control reagent;
drying, for example at a temperature comprised between 35 and 40° C., in particular 37° C., for example during 30 minutes, for example in a ventilated incubator of the previously coated membrane.

In an embodiment of the invention, the process for manufacturing a device for measurement does not comprise any saturation step nor any cleaning step of the membrane after coating.

In an embodiment of the invention, the process for manufacturing a device for measurement, is carried out by means of coating reagents which are capture antibodies, for example monoclonal antibodies. The quantity of reagents deposited must be determined in such a way that well defined coating lines are obtained.

The post-coating drying temperature is determined depending on the molecules constituting the capture reagents of the analyte and the capture control reagents.

The labels (or tracers) of the capture reagents of the analyte test and control, are used without it be required to link them to a matrice (such as latex particles). In other words, the capture reagents are labelled at the end of a coupling process for example by absorption of the label on the capture reagent to provide a conjugate which can then be purified, for example by centrifugation.

The invention in particular proposes a device for carrying out the method, comprising a strip support formed with at least one orifice for depositing the sample and a window for reading a result at test and control zones of the strip, responding to the characteristics described in the present application, and means for capturing signals generated by the labels in the test and control zones, said device being characterized in that it comprises means for detecting the passage of labels into a given zone of the strip at the start of sample migration, means for measuring time, and means for controlling said means for measuring time from detection of the passage of labels into the given zone of the strip, and controlling means for capturing signals after the passage of a predetermined period of time.

Such a device can also be constituted by including the variants features described in the present application.

Advantageously, the means for detecting the passage of labels in said zone of the strip are constituted by the means for capturing signals generated by the labels in the test and control zones.

Said detection and capture means comprise a means for illuminating the strip at a given wavelength and at least one photodetector associated with means for scanning the illuminated zone of the strip.

Said device also comprises means for determining the area of the signals generated by the labels in the test and control zones, and for calculating the ratio of said areas and for determining the quantity of the desired substance in the sample from a calibration table or curve which has already been stored in the memory.

The support for the strip comprises a casing in which the strip is housed and immobilized and which has an upper face comprising at least one opening for detecting the passage of labels at the start of sample migration and for capturing signals generated by the labels in the test and control zones.

Said opening is extended in the direction of sample migration and one of its ends takes in said zone for passage of labels at the start of migration, while its other end takes in the test and control zones.

As will be explained in more detail in the examples, the method of the invention provides an aid to medical diagnosis carried out under emergency conditions to exclude certain diseases, limiting recourse to certain invasive investigations or to expensive analyses.

This is the case, for example, in the field of hemostasis, the diagnosis of venous thromboembolic disease which includes deep vein thromboses and pulmonary embolism.

It is estimated that in France, pulmonary embolism alone could affect close to 100 000 patients per annum, and be responsible for 10000 to 20000 deaths. Thus, it is a serious disease which may involve all categories of patients, whether hospitalized or ambulatory. As it cannot be diagnosed simply by clinical examination, diagnosis involves complementary examination of expensive imagery (sometimes invasive) such as the angioscanner, angiography or scintigraphy, usually associated with a Doppler scan of the lower limbs. Thus, diagnosis requires expensive apparatus and specialized personnel, not always available in care establishments. Further, several examinations may be necessary to exclude with certainty the diagnosis of pulmonary embolism or, in contrast, to confirm it, thus justifying using effective anti-coagulant treatment. However, it should be noted that the diagnosis of pulmonary embolism is only confirmed by exploration in less than one third of ambulatory patients admitted to the emergency services—hence the interest in developing non invasive biological tests that can confirm or reject the diagnosis of a pulmonary embolism and, more generally, of venous thromboembolic disease. The majority of tests have involved coagulation activation tags, in particular for fibrinolysis, especially D-dimers, the specific degradation products of fibrin. The importance of assaying D-dimers to exclude a diagnosis of pulmonary embolism had been clearly identified.

D-dimers appear as degradation products of a fibrin clot under the action of plasmin. The thrombin generated during coagulation activation cleaves the fibrinogen molecule causing, after liberating two small peptides (FPA and FPB), the generation of fibrin monomers which then polymerize (soluble clot). Stabilization of the clot (insoluble clot) is dependent on factor XIII the activated form of which is a transglutaminase which can cross link fibrin molecules involved via their D domains. Activation of the fibrinolytic system will then cause, via plasmin generation, lysis of the thrombus thus constituted. The protease action of plasmin on fibrin results in the liberation of may cleavage fragments of various sizes including D-dimers which appear relatively late compared with the initial thrombotic process. It is none other than an assembly of heterogeneous degradation products on the biochemical scale. It should be noted that plasmin is also capable of cleaving fibrinogen as well as other coagulation factors during pathological activation of the fibrinolytic system. The products derived from fibrinogen or fibrin degradation are collectively known as FDP fibrin/fibrinogen degradation products). Thus, D-dimers are a reflection of lysis by plasmin of polymerized fibrin molecules which have been subjected to the action of factor XIII.

The amount of plasmatic D-dimer is considered to be a good indication of the formation of a clot of fibrin and its lysis. It increases in several clinical cases: deep vein thromboses, pulmonary embolism, disseminated intra vascular coagulation (DIVC), surgery, cancer and cirrhoses, etc. The amount of D-dimers has thus become a very valuable tool used as an aid to the diagnostic exclusion of venous thromboembolic disease (VTED), whether deep vein thrombosis (DVT) or pulmonary embolism (PE).

For this reason, the development of a sufficiently reliable rapid test for quantitative assay independent of user experimentation and environmental conditions is of particular advantage to limit recourse to examinations such as a pulmonary angiography, phlebography or pulmonary scintography.

The assay of D-dimer in a blood sample using the method of the invention is illustrated in the examples below.

In an advantageous embodiment of the invention, illustrated in the examples, the dosage method provides a result characterized by a coefficient of variation of the measure (CV) around the threshold of 500 ng/ml of D-Dimer, which is of the order of 10%, if not from 6 to 8%, if the reading lines of the signal are remote of one further millimeter.

The blood sample which is quantitatively assayed in accordance with the invention is, for example, a plasma sample. A quantity of about 30 μl of plasma may be deposited on the strip to carry out the test. Reading the test leads to the amount of D-dimer, resulting from the ratio obtained between the measurement of the signal for the test analyte and that for the RMC control signal.

The invention also pertains to a kit for carrying out a quantitative method for measurement of at least one analyte of interest in a liquid sample by immunochromatography, comprising:
an inert support on which are immobilized, in distinct zones, different types of compounds termed capture compounds allowing (i) for each capture compound for the analyte, capture of a predetermined analyte of the test sample by a specific binding reaction between said capture compound and an analyte contained in the sample, when said analyte forms a specific bound partner with a conjugate constituted by a reagent carrying a detectable tag, and (ii) for a capture compound for the control reagent, capture of a control reagent carrying a detectable tag;
a conjugate or a mixture of different conjugates, each conjugate being constituted by a reagent that is capable of specifically binding with a predetermined analyte which is being investigated in a biological sample, said conjugate further carrying a detectable tag, said conjugate(s) being contained in a saturated reservoir of conjugates;
a control reagent (RMC: ratiometric measurement control) carrying a detectable tag absorbing at the same wavelength as the tag for the conjugate(s);
for each analyte, a standard curve specific to said analyte, established by assaying a reference analyte associated with a conjugate at a plurality of concentrations and by assaying the control reagent (RMC) at a given concentration under the same conditions, each point on the curve corresponding to the ratio of the signal produced by the conjugate associated with the analyte and captured by the analyte capture compound and the signal produced by the control reagent captured by the capture compound of the control reagent;
one or more supports (backing) to contain the inert supports for carrying out the test;
if appropriate, a case to contain the inert supports and their accessories to constitute a cassette to be placed in the signal reader;
if appropriate, instructions for using the kit to measure the quantity of analyte or analytes.

In particular, the capture compounds are immobilized (coated) onto an inert membrane in the form of a strip, for example a nitrocellulose membrane.

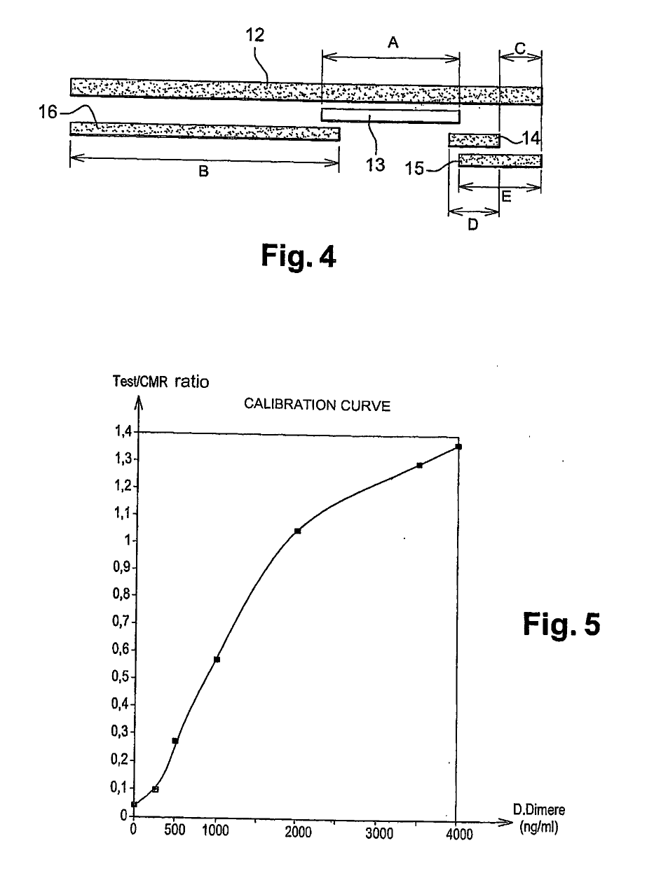

The different parts of the device comprising strips are illustrated in the examples and in FIG. 4.

In a particular implementation of the method or kit of the invention, the capture compounds are capture antibodies, in particular monoclonal antibodies for capturing the analyte and polyclonal antibodies for RMC capture.

The analyte capture antibody is immobilized on the inert support in a concentration of 1.5 to 4 mg/ml, for example about 2±0.1 mg/ml, for example.

The control reagent capture antibody is immobilized on the inert support in a concentration of 0.5 to 2 mg/ml, for example at about 1±0.05 mg/ml, for example.

The invention will be better understood when reading the following description made as an example, with reference to the appended drawings, in which:

FIG. 1 represents very schematically a strip for immunochromatography;

FIG. 2 illustrates the general principle of the dosage according to the invention;

FIG. 3 schematically illustrates the use of a result reading apparatus;

FIG. 4 schematically represents a mode of assembly of a strip of immunochromatography;

FIG. 5 represents a standard curve of calibration:

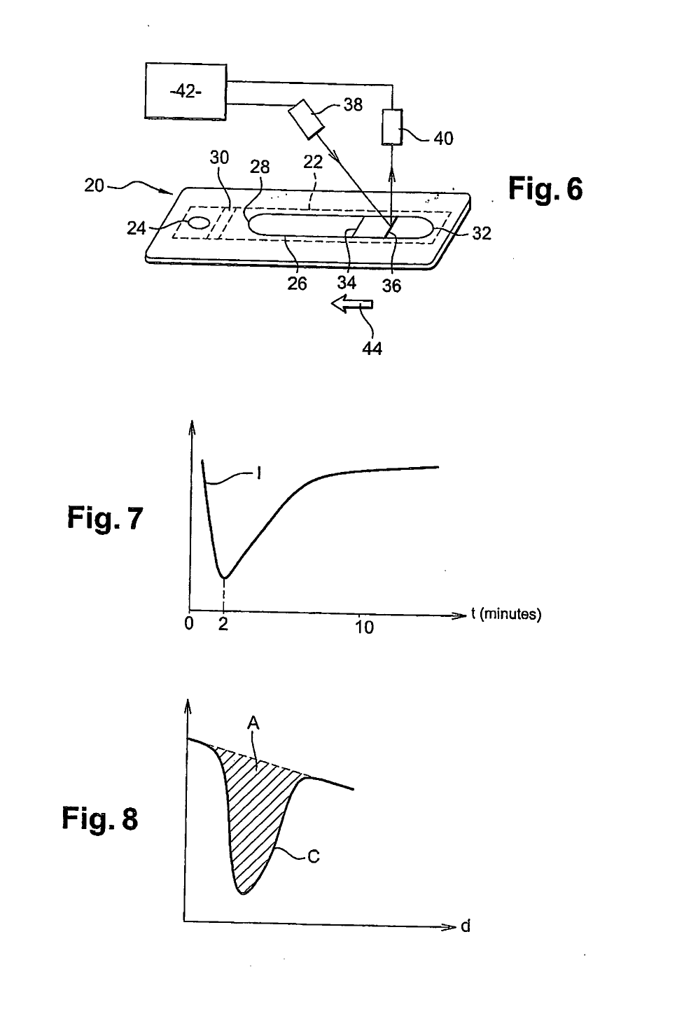

FIG. 6 represents the main components of a device for dosage according to the invention;

FIG. 7 is a graph of variation of the intensity of a light signal captured by a photodetecting device as a function of the time,

FIG. 8 is a graph illustrating the acquisition of a signal generated by labels in a test or in a control zone of the strip.

FIG. 1 is a summary of the principle of strip immunochromatography.

This strip respectively comprises:
a deposition zone 1 on which the sample is deposited; the deposition zone is shown with a reservoir containing the conjugate in the dry form;
a capture zone 2 comprising an immobilized capture antibody specific to the investigated analyte;
a PCZ zone 3 comprising an immobilized capture antibody specific to the conjugate. The excess conjugate which has not reacted with the analyte of the sample migrates along the membrane to the PCZ zone, where it is stopped by the capture antibody;
an absorbant 4 which acts both as a pump and a dump for the immunological reactions triggered by the passage of analyte of the sample over the reactive strip. It acts to increase the absorption capacity of the entire device to finally “clean” the migration membrane on which the immunological reactions resulting in assay have taken place.

FIG. 2 shows the general principle of the device of the invention. In this diagram, it is assumed that only one analyte is being investigated. The membrane 5 respectively comprises:
a deposition zone 1 on which the sample is deposited. In this example, the conjugate and the RMC control reagent are in the dry form in a reservoir 6 in the region of the deposition zone.
a capture zone 2 comprising an immobilized capture antibody specific to the investigated analyte;
a RMC zone 7, comprising an immobilized control capture antibody specific to the tagged control reagent. This control reagent, rendered soluble by depositing the sample on the deposition zone, migrates along the membrane by capillary action to the RMC zone, where it is specifically captured by the control capture reagent. At this RMC zone, then, a signal develops which is completely independent of the concentration of the analyte to be assayed;
an absorbant 4 which acts both as a pump and a dump for the immunological reactions triggered by the passage of analyte of the sample over the reactive strip. It acts to increase the absorption capacity of the entire device to finally “clean” the migration membrane on which the immunological reactions resulting in assay have taken place. Excess conjugate which has not reacted with the analyte of the sample or with the RMC capture reagent migrates along the membrane and then is aspirated by absorbant.

The functioning principle for the results reading device is summarized in FIG. 3.

In this diagram, the reader 8 measures the light which is reflected 9 and transmitted 10 by an immunochromatographic strip 5.

Two distinct sources of light 11 and 11′ are used to illuminate the strip 5.

FIG. 4 is a diagram showing the assembly of the immunochromatography strip in cross section.

The actual dimensions of the supports forming part of the composition of the strip (dimension 7 cm±5 mm) are:
12=backing (adhesive support) (70 mm);
13=coated nitrocellulose membrane (40 mm);
14=reservoir for conjugates (7 mm);
15=filter (16 mm);
16=absorbant (18 mm).

Positions of Coatings:

Absorbant/nitrocellulose membrane=AB=4 mm

Conjugate reservoir/nitrocellulose membrane=CD=1 mm

Filter/conjugate reservoir=DE=6 mm

FIG. 5 illustrates an example of a standard curve for calibration, giving the D-dimer concentration as a function of the test/RMC ratio.

The device of FIG. 6 essentially comprises a casing 20 formed from plastic material in which a strip 22 of porous material is positioned and held, the outline of which is shown as a dotted line, the upper face of the casing 20 comprising an opening 24 for depositing a liquid sample and an optional migration buffer at one end of the strip 22, and a reading window 26 which is elongate in shape which extends, for example, over about half the length of the strip 22 to near its end opposite to that onto which the liquid sample is deposited.

The strip 22 is produced from a material based on nitrocellulose, for example, in which the liquid sample deposited through orifice 24 may migrate by capillary action.

As already described above, the strip 22 may comprise, between the orifice 24 and the first end 28 of the window 26, a zone 30 on which has been deposited, firstly a conjugate intended to form a complex with the substance to be assayed in the liquid sample and secondly, a migration control product.

The portion of the strip appearing in the window 26, close to the second end 32 thereof, comprises a test zone 34 and a control zone 36, shown by transverse lines on the strip on which a test capture reagent and a control capture reagent respectively have been deposited.

The reagent for the test zone or line 34 is intended to capture the complex formed by the conjugate and the substance to be assayed. The control line reagent 36 is intended to capture the control product deposited in said zone 30 of the strip.

As an example, in the case of assaying D-dimer, the conjugate deposited in zone 30 in the dry form is a F(ab)′₂ anti-D-dimer/F(ab)′₂9C3 fragment of the antibody designated 9C3 or 8D2 in the Liatestg D-DI kit from Diagnostica Stago (France), and the control product, also deposited in the dry form, is an anti-biotin antibody or an antibody of IgG of hen; the capture reagent for the test zone 34 is an anti-D-dimer/2.1.16 antibody and the capture reagent for the control zone 36 is a gamma biotin or antibody-IgG of hen, the control product and the capture reagent for this product not having any cross reactivity with the substance to be assayed.

The conjugate and the control product deposited in the zone 30 comprise detection or marking labels which may be of any type, such as particulate labels (gold, latex, carbon, polycarbonate particles, etc) or colorants, fluorescent tracers or enzymatic labels.

In one preferred implementation of the invention, the labels used for the conjugate and the control product are colloidal gold particles which have a light absorption peak at a wavelength of 530 nm such that said labels may be detected by illuminating the strip with a light source 38 emitting at a wavelength of 530 nm or at a wavelength very close thereto and by formation of an image of the illuminated zone at a photodetector or an assembly of photodetectors 40 sensitive to said wavelength.

Preferably, the light source 38 and the photodetector 40 are on the same side of the strip above the window 28 of the casing 20, the light intensity being measured by reflection. In a variant, the light source 38 and the photodetector 40 may be either side of the strip, and the measurement is then made by transmission.

The photodetector 40 or assembly of photodetectors is controlled by data processing means 42 which also control the operation of the light source 48 and receive output signals from the detection means 40. When a single photodetector is used, or a battery of photodetectors aligned over a test or control zone 34, 36, said photodetection means are associated with scanning means which allow them to successively target the various zones of the strip covered by the liquid sample as it migrates. As an example, said scanning means may comprise means for displacing the casing 20 in the direction indicated by the arrow 44 with respect to the detection means 40. It is also possible to use optical scanning means or an array of photodetectors, making the use of a scanning means redundant.

The assay method of the invention is as follows, in the case of assaying D-dimer in a sample of blood plasma:

A fixed volume of plasma is deposited in the orifice 14 of the support 20, then a fixed volume of a migration buffer is deposited in said orifice. The liquid deposited in the orifice 20 is displaced under capillary action in the strip 22 in the direction of its opposite end and passes initially via the zone 30 which comprises the conjugate serving as the test and the control product. The conjugate is dissolved by the sample and by the migration buffer and reacts with the D-dimer to form a complex. Said complex migrates in the strip 22 with the control product by capillary action and thus is displaced towards the first end 28 of the window 26.

During this period, the casing 20 is placed in a reader provided with a light source 38, detection means 40, data processing means 42 and optional scanning means associated with the detection means 40.

In this reader, the first end 28 of the window 26 is illuminated by the light source 38 and observed by the detection means 40. When at the start of migration in the strip 22, the complex and the control product reach the first end 28 of the window 36, their labels are detected by the detection means 40.

The output signal for the detection means 40 corresponding to detection of the labels is shown diagrammatically in FIG. 7, which is a graph showing the variation in the light intensity captured by the detection means 40 as a function of time. The light intensity I captured by the means 40 reduces as soon as the labels appear at the first end 28 of the window 26, passes through a minimum and then increases progressively to a plateau during which measurements are made on the test zone 34 and control zone 36.

The leading edge of this signal at the moment at which the labels appear at the first end 28 of the window is fairly steep and may be used to start a clock by dint of the data processing means 42 which, in conventional manner, are equipped with a clock generating a time signal.

The measuring signal generated by the detection means 40 observing the test zone 34 or the control zone 36 is shown diagrammatically in FIG. 8, where the curve C corresponds to the variation in the light intensity as a function of the scanning distance (d). Said signal is transmitted to the data processing means 42 which calculate the area A of the signal, represented by the hatched portion of FIG. 8.

The quantity of D-dimer present in the sample is determined from the ratio R of the area of the signal measured in the test zone 34 to the area of the signal measured in the control zone 36, and from an established calibration table or curve which directly provides the concentration of D-dimer from the ratio R (FIG. 5).

Detecting the labels at the first end 28 of the window 26 has a certain number of advantages:
the measurement is made in the test and control zones 34, 36 a certain time (for example 10 minutes) after detecting the appearance of the labels at the end 28 of the window 26, said time interval being the same for all samples and not dependent on the conditions for dissolving the conjugate in zone 30 of the strip and for complex formation between said conjugate and the substance to be assayed;
an operator error, such as forgetting to deposit the migration buffer in the orifice 24 of the casing 20 or using a strip 22 which has already been used, can rapidly be detected in the measurement because the signal I of the FIG. 7 would not have the leading edge characteristic of the presence of labels.

### EXAMPLE 1

### Method for Coating or Immobilization on Nitrocellulose Membrane

Equipment

Anti-D-dimer capture antibody to be immobilized: monoclonal 2.1.16 (Stago, France) at a concentration of 2 mg/ml.

RMC capture antibody to be immobilized: goat anti-chicken Ig antibody (Sigma, France) to a concentration of 1 mg/ml.

Coating membrane: nitrocellulose SHF0900405 (Millipore, USA).

Coating buffer: 0.15 M phosphate buffer, pH 7.4.

Antibody solutions distribution equipment: Linomat IV (Camag, Switzerland) or Bio Jet Quanti 3000 (Bio dot, USA).

Method

The capture antibodies and proteins were coated onto (immobilized on) an inert support using the method briefly summarized below. 1 μg per test of test 2.1.16 capture antibody and 0.25 μg per test of goat anti-chicken RMC capture antibody were distributed, using a Linomat or Bio Jet Quanti apparatus (by vaporization), onto the nitrocellulose membrane, pre-cut into a form that was suitable for the test.

After depositing the antibodies onto the membrane leading to the absorption of the antibodies on the membrane, it was immediately dried in a ventilated oven for 30 minutes at 37° C. The membrane, coated with the antibodies, was stored in a sealed aluminum pouch at ambient temperature (22° C.-25° C.).

### EXAMPLE 2

### Process for Coupling Antibody with Colloidal Gold Particles

Equipment

Anti-D-dimer monoclonal antibody: F(ab)′₂ 9C3 fragments (Stago, France).

RMC revealing antibody: chicken IgG (Sigma, France).

Tetrachloroauric acid (Sigma, France).

Tri sodium citrate (Prolabo, France.

PEG₂₀₀₀₀ (Prolabo, France).

Borate buffer, 2 mM.

K₂CO₃ solution, 0.2 M.

Conjugate reservoir: PT-R5 (MDI, India).

Method

Preparation of Colloidal Gold Particles

Particles of colloidal gold, 50 nm in size, were prepared using a slight modification of the Frens-Tinglu method. This method will be briefly summarized.

Add 2 ml of 1% tetrachloroauric acid to 198 ml of ultrapure water in a clean flask.

Place a magnetic stirrer in the flask and heat to boiling point.

Add 275 μl of 10% tri sodium citrate solution, continuing the stirring. Continue heating.

After a violet color appears, heat for a further 10 minutes.

Allow to cool to ambient temperature (22-25° C.).

Store the colloidal gold particles at 4° C. protected from light in a smoked glass bottle.

The prepared particles are characterized: measure the maximum wavelength and OD at this wavelength, to measure the pH.

Preparation of Test Anti-D-Dimer—Colloidal Gold Particles Conjugate.

This preparation comprises several steps.

a) Preparation of Antibody for Coupling

The anti-D-dimer 9C3 (fragment F(ab)′₂, Stago) antibody is coupled to gold particles using the following protocol.

Dilute the antibody in 2 mM borate, pH 6.5, to produce a final concentration of 1 mg/ml then dialyze it against that buffer for 3 hours at ambient temperature (22-25° C.).

Centrifuge the antibody solution for 15 minutes at 5000 rpm at 4° C. After recovering the supernatant containing the antibody, verify its concentration by spectrophotometric measurement at 280 nm prior to coupling.

b) Preparation of Colloidal Gold for Coupling

Adjust the OD_{530 nm} of colloidal gold particles to 1 by adding Milli-Q water.

Adjust the pH of the solution of colloidal gold particles to 6.5±0.1 by adding 0.2 M K₂CO₃.

c) Coupling by Adsorption

Mix the antibody solution with 0.2 mg/ml of the solution of colloidal gold particles in a ratio of 1/10 (antibody volume/gold particles volume), for example 1.85 ml of antibody for 18.5 ml of colloidal gold particles.

Stir for two minutes at ambient temperature (22-25° C.).

d) Stabilization after Coupling and Recovery of Conjugate

Add 1.2 ml of 1% PEG₂₀₀₀₀ and vortex for 2 minutes at ambient temperature (22-25° C.).

Add 2.4 ml of 1% BSA and vortex for 2 minutes at ambient temperature (22-25° C.).

Centrifuge for 1 hour at 9000 rpm, at 4° C.

Eliminate the supernatant and recover the residue in 20 mM Tris buffer, pH 8.2, PEG₂₀₀₀₀, 1% BSA, 0.09% sodium azide.

Filter the recovered conjugate over 0.2 μm.

The conjugate is characterized spectrally.

Measure λₘₐₓ and ODₘₐₓ.

Preparation of chicken IgG—colloidal gold particles RMC conjugate.

The same steps are followed to prepare the RMC conjugate. The difference essentially lies in the coupling concentration and the coupling pH: 0.1 mg/ml and 9.5.

### EXAMPLE 3

### Saturation of Conjugate Reservoir and Deposition of Conjugate

Immerse the PTR5 film (MDI, India) in the saturation solution (PBS, 1% milk, 5% sucrose) in an amount of 1 ml of solution per test. Leave for 1 hour at ambient temperature, with stirring.

After eliminating the saturation solution, dry the reservoir film for 3 h at 37° C. in a ventilated oven.

The mixture of two conjugates (test and RMC) is deposited on the saturated conjugate reservoir in an amount of 8 μl per cm, i.e. 4 μl per test, the conjugate reservoir being 0.5 cm wide. Deposition is carried out using the Air Jet system (Bio dot).

After deposition, the conjugate reservoir is dried for 1 hour at 37° C. in a ventilated oven.

### EXAMPLE 4

### D-Dimer Assay Test Cassette

### Immunochromatographic Strip

The different constituent elements of the strip employed were as follows:
absorbant (17 CHR, Whatman, USA);
sample filter (GF 3596, Schleicher & Schüell, Germany);
nitrocellulose membrane (SHF 0900405, Millipore, USA), coated;
reservoir (PT-R5, MDI, India) of deposited conjugate.

These were assembled on a rigid inert support termed the “backing” (010 white polyester GL-187, G&L, USA).

The assembly diagram is shown in FIG. 4.

After assembly, the 5 mm wide and 70 mm long strips were cut out with a cutter (Bio dot, USA).

Each strip was inserted into a plastic case similar to that illustrated in FIG. 6.

After inserting the strip, the plastic case was closed and packed into an aluminum pouch (Soplaril, France) with a desiccant (BO54/Indice 1, Airsec, France) then stored at 2-8° C.

### EXAMPLE 5

### Assay of D-Dimer Using the Method of the Invention

Equipment

Migration buffer: 0.5% PBS casein, 0.1% triton X100;

D-dimer calibration agent: D-dimers prepared from human plasma (Stago, France).

Strip reader: readers: Rapi-kit (77 Elektronika, Hungary).

Method

Operating Protocol

The following protocol was used:
allow the strips, buffers, samples to stabilize at ambient temperature (22-25° C.);
deposit 15 μl of D-dimer calibration agent or plasma sample on the sample well of the plastic case;
add 150 μl of migration buffer;
leave for 10 minutes at ambient temperature;
read the result with an immunochromatography strip reader (77 Elektronika).

All tests were carried out in duplicate.

Obtaining and Interpreting Results

Firstly, the standard curve was established from assaying the D-dimer calibration agent stabilized after reconstitution at different concentrations from 0 to 4000 ng/ml. For each assay, the two signals, test and RMC, were measured by the reader and the test/RMC ratio was calculated.

By transferring this ratio into a function of the concentration of D-dimer calibration agent, the standard curve was constructed. It was valid for a given batch of test cassette. The RMC signal was selected by the skilled person to be in the rising portion of the signal/complex curve formed between the RMC capture reagent and the RMC was around 4000±500.

Secondly, the ratio measured for each assay of plasma with an unknown D-dimer concentration was transferred onto said standard curve. It was then possible to translate the ratio measured into the concentration of investigated analyte.

The steps for producing the calibration curve were as follows:
a) reconstitute the calibration agent approximately (4500 ng/ml) with MilliQ water;
b) allow the calibration agent to stabilize for 30 minutes. The calibration agent is stable for 4 hours at AT once it had been reconstituted;
c) dilute the calibration agent in the migration buffer to obtain the following D-dimer concentrations:
   250, 500, 1000, 2000 and 4000 ng/ml;
d) carry out the tests in duplicate;
e) deposit 15 μl of sample in the sample well (for point 0 of the calibration, 15 μl of migration buffer is deposited);
f) add 150 μl of migration buffer (10 mM PBS-0.5% casein-0.1% triton×100-0.095% sodium azide);
g) wait 10 minutes at ambient temperature;
h) read the results using the reader;
i) transfer the results to the results films;
j) trace the calibration curve [amount of D-dimer=f(test/RMC ratio)];
k) assay plasma and control following the protocol from a to i.

### Standard Curve Results

### EXAMPLE

| | | | | | | Mean |
|---|---|---|---|---|---|---|
| Calibration | D-dimers | test | RMC | Test | Test/RMC | test/RMC |
| agent | (ng/ml) | no | area | area | ratio | ratio |
| 1 | 0 | 1 | 4664 | 116 | 0.025 | 0.027 |
| | | 2 | 4503 | 132 | 0.029 | |
| 2 | 250 | 1 | 4392 | 431 | 0.098 | 0.098 |
| | | 2 | 4953 | 483 | 0.098 | |
| 3 | 500 | 1 | 4529 | 1127 | 0.249 | 0.267 |
| | | 2 | 3744 | 1068 | 0.285 | |
| 4 | 1000 | 1 | 4234 | 2368 | 0.559 | 0.573 |
| | | 2 | 4554 | 2669 | 0.586 | |
| 5 | 2000 | 1 | 4992 | 5335 | 1.069 | 1.056 |
| | | 2 | 4551 | 4752 | 1.044 | |
| 6 | 4000 | 1 | 5256 | 6866 | 1.306 | 1.376 |
| | | 2 | 4835 | 6994 | 1.447 | |

### Plasma Assay Example

| | | | | | RMC | Comparison |
|---|---|---|---|---|---|---|
| | | | | | method | method |
| Plasma | Test | RMC | Test | Test/RMC | assay | assay |
| name | no | area | area | ratio | (ng/ml) | (ng/ml) |
| 0105290294 | 1 | 3492 | 369 | 0.106 | 280 | 310 |
| | 2 | 4963 | 606 | 0.122 | | |
| 0107020181 | 1 | 4821 | 5493 | 1.139 | 2750 | 2600 |
| | 2 | 4622 | 5844 | 1.264 | | |
| 0105100264 | 1 | 5009 | 5694 | 1.137 | 2325 | 2250 |
| | 2 | 3223 | 3638 | 1.129 | | |
| 0105290418 | 1 | 5227 | 4610 | 0.882 | 1420 | 3370 |
| | 2 | 4059 | 3147 | 0.775 | | |
| 0105050112 | 1 | 3930 | 5326 | 1.355 | 3400 | >4000 |
| | 2 | 4299 | 5402 | 1.257 | | |
| 0106160147 | 1 | 4778 | 3848 | 0.805 | 1450 | 1490 |
| | 2 | 5583 | 4844 | 0.868 | | |
| 0106190137 | 1 | 4646 | 3314 | 0.713 | 1300 | 1830 |
| | 2 | 5010 | 3988 | 0.796 | | |
| 0107060177 | 1 | 4293 | 5252 | 1.223 | 3450 | 3850 |
| | 2 | 3587 | 4963 | 1.384 | | |
| 0105240148 | 1 | 4299 | 2637 | 0.613 | 1125 | 1080 |
| | 2 | 3805 | 2567 | 0.675 | | |
| 0105220259 | 1 | 5251 | 853 | 0.162 | 375 | 260 |
| | 2 | 4166 | 796 | 0.191 | | |
| 0105250240 | 1 | 4462 | 2848 | 0.638 | 1100 | 780 |
| | 2 | 5803 | 3633 | 0.626 | | |
| 0105250267 | 1 | 3921 | 1398 | 0.357 | 660 | 580 |
| | 2 | 4754 | 1794 | 0.377 | | |
| 0105300196 | 1 | 3718 | 5575 | 1.499 | 3625 | >4000 |
| | 2 | 4142 | 4839 | 1.168 | | |
| 0106070275 | 1 | 4575 | 3392 | 0.741 | 1250 | 1560 |
| | 2 | 3551 | 2856 | 0.804 | | |
| 0106010160 | 1 | 5015 | 2687 | 0.536 | 1050 | 1130 |
| | 2 | 4604 | 3039 | 0.660 | | |
| 9740 | 1 | 4296 | 4631 | 1.078 | 2400 | 2078 |
| | 2 | 3930 | 4693 | 1.194 | | |
| 9432 | 1 | 4378 | 4963 | 1.134 | 2100 | 2405 |
| | 2 | 4873 | 4982 | 1.022 | | |
| 7355 | 1 | 3261 | 4574 | 1.403 | >4000 | 3391 |
| | 2 | 3216 | 4848 | 1.507 | | |
| 8199 | 1 | 4439 | 5950 | 1.340 | >4000 | 5534 |
| | 2 | 3980 | 6230 | 1.565 | | |
| 9666 | 1 | 3370 | 5212 | 1.547 | >4000 | 31093 |
| | 2 | 4614 | 6539 | 1.417 | | |
| 8967 | 1 | 3845 | 565 | 0.147 | 380 | 412 |
| | 2 | 4261 | 792 | 0.186 | | |
| 6616 | 1 | 3571 | 6111 | 1.711 | >4000 | 9041 |
| | 2 | 4996 | 7021 | 1.405 | | |

The assay results obtained with the method of the invention were compared with those obtained with a reference method based on ELISA, Asserachrom DDimère (Stago, France).

We can see that the results obtained with the method of the invention are very close to those of the reference method.

Thus, we have a method which allows quantitative determination which can provide results equivalent to those of the reference method. The subject matter of the invention, RMC, allows the variations inherent to the immunochromatographic technique (variations due to supports and the sample, etc) to be weighted.

It is possible to estimate this weighting from the results obtained.

Effect of RMC Weighting

Each assay was carried out in duplicate. The weighting effect due to RMC was estimated from the absolute deviation between the two values obtained for each assay. For a perfect theoretical weighting system, the deviation between the two values of the same assay has to be equal to 0.

| Calibration | Test signal | |
|---|---|---|
| agent or plasma | absolute difference | Absolute difference ratio |
| 0 | 16 | 0.004 |
| 250 | 52 | 0.001 |
| 500 | 59 | 0.036 |
| 100 | 301 | 0.027 |
| 2000 | 583 | 0.025 |
| 4000 | 128 | 0.140 |
| 0105290294 | 237 | 0.016 |
| 0107020181 | 351 | 0.125 |
| 0105100264 | 2056 | 0.008 |
| 0105290418 | 1463 | 0.107 |
| 0105050112 | 76 | 0.099 |
| 0106160147 | 996 | 0.062 |
| 0106190137 | 674 | 0.083 |
| 0107060177 | 289 | 0.160 |
| 0105240148 | 70 | 0.061 |
| 0105220259 | 57 | 0.029 |
| 0105250240 | 785 | 0.012 |
| 0105250267 | 396 | 0.021 |
| 0105300196 | 736 | 0.331 |
| 0106070275 | 536 | 0.063 |
| 0106010160 | 352 | 0.124 |
| 9740 | 62 | 0.116 |
| 9432 | 19 | 0.111 |
| 7355 | 274 | 0.105 |
| 8199 | 280 | 0.225 |
| 9666 | 1327 | 0.129 |
| 8967 | 227 | 0.039 |
| 6616 | 910 | 0.306 |
| Mean | 475.4 | 0.092 |

These results show that the smallest differences are obtained only with the system of the invention.

In contrast, in the absence of RMC, and thus using only a single test signal, assay is possible but the uncertainty in the measurement is greater.

The weighting effect was further demonstrated by repeatability tests. In this case, the same sample was assayed 21 times using the method of the invention.

REPEATABILITY TEST
Plasma 1

| | Plasma 1 | | | |
|---|---|---|---|---|
| | | | Test/ | DDimer assay |
| Tests | Test | RMC | RMC ratio | (ng/ml) |
| 1 | 2063 | 5699 | 0.36 | 650 |
| 2 | 2518 | 6103 | 0.41 | 725 |
| 3 | 2395 | 5822 | 0.41 | 725 |
| 4 | 1997 | 4451 | 0.45 | 775 |
| 5 | 2236 | 4637 | 0.48 | 770 |
| 6 | 1559 | 4314 | 0.36 | 650 |
| 7 | 2186 | 4955 | 0.44 | 760 |
| 8 | 2368 | 5799 | 0.41 | 725 |
| 9 | 1911 | 5310 | 0.36 | 650 |
| 10 | 1939 | 5508 | 0.35 | 630 |
| 11 | 1608 | 4523 | 0.36 | 650 |
| 12 | 2067 | 5247 | 0.39 | 680 |
| 13 | 1989 | 5276 | 0.38 | 675 |
| 14 | 1442 | 4383 | 0.33 | 600 |
| 15 | 2312 | 5801 | 0.40 | 700 |
| 16 | 2411 | 5482 | 0.44 | 760 |
| 17 | 2228 | 5471 | 0.41 | 725 |
| 18 | 2392 | 5560 | 0.43 | 750 |
| 19 | 1872 | 5652 | 0.33 | 600 |
| 20 | 2008 | 5351 | 0.38 | 675 |
| 21 | 2043 | 4718 | 0.43 | 750 |
| Mean | 2073.52381 | 5241.04762 | 0.396 | 696.429 |
| Standard | 292.10 | 540.45 | 0.04 | 55.46 |
| deviation | | | | |
| C.V. (%) | 14.09 | 10.31 | 10.45 | 7.96 |

With these repeated assays, the method of the invention allowed the variations to be weighted. A gain of 4% to 6% in variation was observed, compared with the test signal alone.

1. A method for the quantitative measurement of at least one analyte of interest in a liquid sample by immunochromatography, said method comprising the following steps:
a) providing an immunochromatography device comprising a solid support in the form of a strip, said support comprising a sample application zone, a migration zone, a specific capture zone for each of the investigated analytes and a ratiometric measurement control zone (RMC), the specific capture zone or zones each comprising a capture reagent immobilized on the support, said capture reagent forming a specific bound pair with one of the investigated analytes, and said RMC zone comprising a control capture reagent immobilized on the support, said reagent forming a specific bound pair with a control reagent carrying a detectable tag;
b) bringing the sample into contact with a control reagent carrying a detectable tag and with a conjugate or a mixture of conjugates, each conjugate being constituted by a reagent forming a specific bound partner with one of the investigated analytes and carrying a detectable tag absorbing at the same wavelength as the tag for the control reagent;
c) depositing the sample which has been brought into the presence of the control reagent and the conjugate or mixture of conjugates on the application zone of the support;
d) maintaining the device under conditions sufficient to allow the investigated analyte or analytes to form bound pairs with the specific conjugate or conjugates and to allow transport of the investigated analyte or analytes and the control reagent by capillary action along the solid support to each of the specific capture zones for the investigated analyte or analytes and to the RMC zone;
e) maintaining the device under conditions sufficient to allow further binding of the investigated analyte or analytes with the capture reagent or reagents in the capture zone or zones, and binding of the control reagent with the control capture reagent at the RMC zone;
f) measuring the intensity of the test signal produced by the conjugate bound to the investigated analyte at each of the capture zones and that of the RMC control signal produced by the control reagent at the RMC zone, whereby the quantity of each analyte of interest in the sample is directly proportional to the ratio of each test signal to the RMC control signal.

2. A method for the quantitative measurement of at least one analyte of interest in a liquid sample by immunochromatography, said method comprising the following steps:
a) providing an immunochromatography device comprising a solid support in the form of a strip, said support comprising a sample application zone, a migration zone, a specific capture zone for each of the investigated analytes and a ratiometric measurement control zone (RMC), said application zone comprising a reservoir containing a control reagent carrying a detectable tag and a conjugate or a mixture of conjugates, each conjugate being constituted by a reagent that is capable of forming a specific bound partner with one of the investigated analytes and carrying a detectable tag absorbing at the same wavelength as the control reagent tag, and said RMC zone comprising a control capture reagent immobilized on the support, said reagent forming a specific bound pair with the control reagent carrying a detectable tag;
b) depositing the sample on the application zone of the support;
c) maintaining the device under conditions sufficient to allow the investigated analyte or analytes to form bound pairs with the specific conjugate or conjugates and to allow transport of the investigated analyte or analytes and the control reagent by capillary action along the solid support to each of the specific capture zones for the investigated analyte or analytes and to the RMC zone;
d) maintaining the device under conditions sufficient to allow further binding of the investigated analyte or analytes with the capture reagent or reagents at the capture zone or zones, and binding of the control reagent with the control capture reagent at the RMC zone;
e) measuring the intensity of the test signal produced by the conjugate fixed to the investigated analyte at each of the capture zones and that of the RMC control signal produced by the control reagent at the RMC zone, whereby the quantity (or ratio) of each analyte of interest in the sample is directly proportional to the ratio of each test signal to the RMC control signal.

3. A method according to claim 2, wherein the conjugate or mixture of conjugates and the RMC control reagent are used in the dry form.

4. A method according to claim 1 or claim 2, wherein the control reagent captured in the region of the RMC zone and the investigated analytes have no cross reactivity.

5. A method according to claim 1 or claim 2, wherein, when an assay is carried out on a sample of human origin, the RMC is selected from molecules of animal origin.

6. A method according to claim 5, wherein the control reagent is selected from immunoglobulins of animal origin and its capture reagent is an antibody directed against the animal species of said control reagent.

7. A method according to claim 1 or claim 2, wherein the bound pair formed between the RMC control reagent and its specific capture reagent is of the antigen/antibody type.

8. A method according to claim 1 or claim 2, wherein the tag used on the conjugate or conjugate mixture is that used on the RMC control reagent absorbing at the same wavelength.

9. A method according to claim 8, wherein the tag used on the conjugate or conjugate mixture and that used on the RMC control reagents are identical.

10. A method according to claim 8, wherein the tag used is of the particulate type.

11. A method according to claim 10, wherein the tag used is colloidal gold.

12. A method according to claim 1 or claim 2, wherein the test and RMC signals are read using a strip reader.

13. A method according to claim 12, wherein the measurement is carried out by reflectance or transmittance.

14. A method according to claim 1 or claim 2, wherein the test/RMC signal ratio is compared with a standard calibration curve established from an assay of the reference analyte at a plurality of concentrations and a RMC assay, each point on the calibration curve corresponding to an amount of analyte which is directly proportional to the analyte signal/RMC signal ratio.

15. A method according to claim 1 or claim 2, wherein it furthermore consists of automatically detecting the passage of said labels into a given zone of the strip at the start of sample migration, automatically starting counting of a predetermined period of time to detection of said passage and, at the end of said period of time, automatically commencing acquisition of signals generated by the labels in the test and control zones of the strip.

16. A method for quantitative assay by immunochromatography of a substance present in a liquid sample, consisting of bringing the liquid sample into contact with a conjugate which can react with the substance to be assayed to form a complex, and with a control product, the conjugate and the control product comprising detection labels, migrating the sample under capillary action with the conjugate and the control product in a strip of porous material comprising a test zone which contains a reagent which can react with the complex and a control zone which contains a reagent which can react with the control product, detecting the signals generated by the labels in the test zone and in the control zone, producing the ratio of said signals and deducing therefrom the quantity of the substance to be assayed present in the sample, wherein it also consists of automatically detecting the passage of said labels into a given zone of the strip at the start of sample migration, automatically starting counting of a predetermined period of time to detection of said passage and, at the end of said period of time, automatically commencing acquisition of signals generated by the labels in the test and control zones of the strip.

17. A method according to any one of claims 1, 2 and 16, wherein it comprises the measure of the areas A of the signals generated by the labels in the test and control zones, calculating the ratio of said areas and deducing from said ratio the quantity of substance present in the sample.

18. A method according to claim 16, wherein the labels have a light absorption peak at a given wavelength and the method consists of illuminating the strip at this wavelength, measuring the intensity of the light reflected or diffused by the labels at this wavelength using at least one photodetector or an assembly of photodetectors, and processing the signals emitted by said photodetector or photodetectors to obtain the areas A of the signals generated by the labels.

19. A method according to claim 18, wherein it consists of using an array of photodetectors to capture the signals generated by the labels at different points of the strip.

20. A method according to claim 18, wherein it consists of using a photodetector associated with means for scanning the strip to capture the signals generated by the labels as a function of the scanning distance and to determine the areas A of said signals.

21. A method according to any one of claims 1, 2 and 16, wherein the period of time between detecting the passage of the labels into the given zone at the start of sample migration and acquisition of signals in the test and control zones is about 10 minutes.

22. The method according to any one of claims 1, 2 and 16, wherein said analyte is D-dimer, and said sample is a blood sample.

23. The method according to claim 22, wherein said blood sample is a plasma sample.

24. The method according to claim 22, wherein said method allows for exclusion diagnosis of venous thromboembolic disease.

25. A kit for carrying out a quantitative method for measurement of at least one analyte of interest in a liquid sample by immunochromatography, comprising:
a) an inert support on which are immobilized, in distinct zones, different types of compounds termed capture compounds allowing (i) for each capture compound for the analyte, capture of a predetermined analyte of the test sample by a specific binding reaction between said capture compound and an analyte contained in the sample, when said analyte forms a specific bound partner with a conjugate constituted by a reagent carrying a detectable tag, and (ii) for a capture compound for the control reagent, capture of a control reagent carrying a detectable tag;
b) a conjugate or a mixture of different conjugates, each conjugate being constituted by a reagent that is capable of specifically binding with a predetermined analyte which is being investigated in a biological sample, said conjugate further carrying a detectable tag, said conjugate(s) being contained in a saturated reservoir of conjugates;
c) a control reagent (RMC: ratiometric measurement control) carrying a detectable tag absorbing at the same wavelength as the tag for the conjugate(s);
d) for each analyte, a standard curve specific to said analyte, established by assaying a reference analyte associated with a conjugate at a plurality of concentrations and by assaying the control reagent (RMC) at a given concentration under the same conditions, each point on the curve corresponding to the ratio of the signal produced by the conjugate associated with the analyte captured by the analyte capture compound to the signal produced by the control reagent captured by the capture compound of the control reagent;
e) one or more supports (backing) to contain the inert supports for carrying out the test;
f) if appropriate, a case to contain the inert supports and their accessories to constitute a cassette to be placed in the signal reader;
g) if appropriate, instructions for using the kit to measure the quantity of analyte or analytes.

26. A kit according to claim 25, in which the inert support is a nitrocellulose membrane.

27. A kit according to claim 25, in which the capture compounds are capture antibodies, in particular monoclonal antibodies to capture the analyte, and polyclonal antibodies for RMC capture.

28. A kit according to claim 27, in which the capture antibody is immobilized on the inert support in a concentration of 2±0.1 mg/ml.

29. A kit according to claim 25, in which the RMC capture antibody is an IgG bound to biotin in a concentration of 1±0.05 mg/ml.

30. A kit according to claim 25, in which the zones for depositing the various capture compounds are separated from each other by about 5±0.5 mm.

31. A kit according to claim 25, in which the investigated analyte is D-dimer, the analyte capture compound is an anti-D-dimer monoclonal antibody, and the standard calibration curve is established for amounts of D-dimers of between 0 and 4000 ng/ml and a value of the RMC control corresponding to the range 1200-1600 ng/ml of D-dimers.

32. A device for carrying out the method according to any one of claims 1, 2 and 16, comprising a strip support formed with at least one orifice for depositing the sample and a window for reading a result at test and control zones of the strip, and means for capturing signals generated by the labels in the test and control zones, wherein it comprises means for detecting the passage of labels into a given zone of the strip at the start of sample migration, means for measuring time, and means for controlling said means for measuring time from detection of the passage of labels into the given zone of the strip, and for controlling means for capturing signals generated by the labels in the test and control zones after the passage of a predetermined period of time.

33. A device according to claim 32, wherein the means for detecting the passage of labels are constituted by the means for capturing signals generated by the labels in the test and control zones.

34. A device according to claim 33, wherein the detection and capture means comprise a means for illuminating the labels at a given wavelength and at least one photodetector associated with means for scanning the illuminated zone of the strip.

35. A device according to claim 32, wherein it comprises means for determining the area A of the signals generated by the labels in the test and control zones, and means for calculating the ratio of said areas

36. A device according to claim 32, wherein support for the strip comprises a casing in which the strip is housed and immobilized, which has an upper face comprising at least one opening for detecting the passage of labels at the start of sample migration and for capturing signals generated by the labels in the test and control zones.

37. A device according to claim 36, wherein opening is extended in the direction of sample migration and one of its ends takes in said zone for detecting the passage of labels at the start of migration, its other end taking in the test and control zones.
